(19) United States
(12) Patent Application Publication
(10) Pub. No.: US 2002/0016607 A1
(43) Pub. Date: Feb. 7, 2002

(54) Medical device comprising an evertable sleeve

(75) Inventors: Frank Bonadio, Bray (IE); Derek William Young, Blackrock (IE); Alan Reid, Dublin (IE)

Correspondence Address: Finnegan, Henderson, Farabow,, Garrett & Dunner, L.L.P., 1300 I Street, N.W., Washington, DC, 20005-3315, US

(73) Assignee: Atropos Limited

(21) Appl. No.: 09/867,593

(22) Filed: May 31, 2001

Related U.S. Application Data
(63) Continuation of application No. PCT/IE99/00126, filed on Dec. 1, 1999.

(30) Foreign Application Priority Data
Dec. 1, 1998 (IE) 981000
Feb. 15, 1999 (IE) 990109
Feb. 15, 1999 (IE) 990110

(51) Int. Cl. A61M 29/00

(52) U.S. Cl. 606/192

(57) ABSTRACT

A medical device having an introducer for introducing an object such as an instrument through a body opening such as the throat. The device has a tubular sleeve of pliable plastics material which is turned axially back on itself to define inner an outer sleeves sections. The inner sleeve section defines an inner lumen and the sleeve may be twisted to centralise the lumen. The sleeve is pre-shaped to define a desired non-linear shape. A chamber between the inner and outer sleeve section is inflatable. The device may also be deployed remotely for example, for balloon angioplasty.

### INTRODUCTION

The invention relates to a medical device, particularly for use in minimally invasive and endoluminal surgical and medical techniques. In particular the invention relates to an introducer for introducing an instrument into the body through an opening. More specifically, the invention relates to a device to assist in the introduction of endoscopic devices into the lumen of a natural bodily orifice, in particular the rectum and colon.

The introduction of an instrument or the like through a body opening such as the throat or rectum is traumatic for the patient and difficult for the medical practitioner as such openings lead into a complex passageway(s) through which the instruments must be passed. Therefore great skill and experience is required.

### BACKGROUND OF THE INVENTION

The practise of gastroenterology has been much improved due to the development of the fiber-optic endoscope. Modern endoscopes consist of a control section attached to a long flexible shaft with a steerable tip. The flexible shaft carries several tubes for light, air, water and suction. In some cases a biopsy channel with a larger bore to allow therapeutic procedures to be performed is included. Light is transmitted through non-coherent fiber-optic bundles. Older scopes used coherent fiber-optic bundles for transmission of the image but these are largely obsolete now and video-endoscopes are the norm. These use fiber-optic bundles for light transmission only and use a CCD TV camera for image acquisition. The camera output is then transmitted through wire pairs.

Endoscopes use a torque control mechanism that allows the endoscope to be steered through the passage of interest using control wheels on the handle at the proximal end. Steering is achieved by turning control knobs on the control section of the endoscope. There are usually two knobs, one for lateral control and another for vertical control. These control wheels are attached to guide wires that run up through the endoscope and are attached to the tip.

An endoscope is typically 100-150 centimeters long and may be inserted into either end of the digestive system. Generally the devices have specific design features adapted to the bodily opening through which the endoscope is inserted. The endoscope is pushed from the bottom and guided through tortuous passages using external manipulation.

In the upper gastrointestinal tract the insertion of the device presents relatively little difficulty. This is due to the short length of the upper GI tract and the relatively straight anatomy. Difficulty is encountered when attempting to advance into the proximal end of the small intestine for example to examine the pancreatoduodenal junction.

In the lower GI tract the picture is quite different. The lower GI tract is made up of the rectum and the large intestine or colon. The colon, in a textbook arrangement of its anatomy, extends upwards from the lower right quadrant, traverses the width of the body just below the diaphragm, extends downwards along the left side of the abdomen and then loops in a retrograde manner before linking up with the rectum and the anus. Even with this textbook arrangement, the large intestine is difficult to cannulate with a flexible endoscope due to the flexible nature of the endoscope shaft and the floppy nature of the colon. It is even more difficult with the more realistic lumen anatomies. In some people the sigmoid colon can be very long and is unfixed, except by its mesentery, and so can be extremely difficult to cannulate due to its predisposition to form loops when an endoscope is pushed through it. Some anatomical landmarks, such as the rectosigmoidal junction, the splenic flexure and the hepatic flexure, are difficult to pass through simply because of their tortuous nature. Problems traversing these areas are exacerbated by looping of the endoscope in the sigmoid colon.

Endoscopy is a difficult technique that can only be mastered after performing many hundreds of examinations. The ability to speedily cannulate the bowel and traverse the entire colon all the way to the caecum is a skill that is only enjoyed by a minority of endoscopists. Published research on the subject of difficulty encountered in endoscopy shows that the procedure fails in up to 15% of cases where failure is defined as inability to reach or visualise the caecum. Up to 35% of cases are considered to be difficult as defined by extended duration of the procedure and experience of pain by the patient. Other research shows that up to 29% of cases are considered to be technically difficult.

Several devices have been described in the prior art to assist in the practise of lower GI endoscopy.

U.S. Pat. No. 3,805,770 describes an endoscope guide and lubricating means comprising a cylindrical spongy member to guide and lubricate the endoscope as it enters the anus. This device however does not address the problems associated with looping of the scope in the sigmoid colon and resulting problems crossing the splenic and hepatic flexures.

U.S. Pat. No. 4,207,872 device a sleeve device for positioning on the end of an endoscope to assist it in advancing through a body passage. The device has protrusions extending perpendicularly from the sleeve that may be expanded and retracted using fluid pressure. Upon repetitious expansion and retraction of the protrusions using pulsing pressure within the device, the device assists in advancing the scope along the body passage. There is likely to be considerable internal friction between the device and the inner wall of the body passage.

U.S. Pat. No. 4,321,915 describes a device consisting of an everting tube which includes a pressure and evacuation valve to allow eversion and retraction of the tube. The tube is slidably attached to a fiber-optic bundle for vision. Whilst this device allows a means of vision to be advanced up the confines of a body passage it cannot be used with existing endoscopes because of its size. It is proposed for use in small narrow bore tubes such as the urethra. There is a similar problem with the device disclosed in U.S. Pat. No. 4,615,331. This device consists of an everting tube which has a plurality of folds which telescope open as the everting tube advances.

U.S. Pat. No. 4,676,228 describes a device that is removably attached to the front end of an endoscope to assist in pulling the scope through the colon. It consists of two inflatable cuff sections that are alternatively inflated and deflated whilst being moved towards and away from each other. In this way the endoscope is dragged up through the colon in short steps. This procedure is lengthy and complex. In addition, there is considerable friction between the colon and the scope.

U.S. Pat. No. 4,971,033 describes a flexible endoscope with a working channel designed to cause the tube to stiffen when fluid pressure is applied to the channel. The channel takes up space in the endoscope that is more appropriately used for either vision, suction, insufflation or tissue sampling. In addition, an endoscope is stiffened along a particular section or along its entire length which exacerbates the difficulty of passing the endoscope though the floppy sigmoid colon.

U.S. Pat. No. 5,045,070 describes an everting tube for entering body cavitys and depoloying a tube for administration of drugs or therapy through the tube. The device is designed for introducing a channel attached to the tube and is not designed for introducing a removable endoscope or the like.

WO-A-97/32515 describes a semi-toroidal tube for introducing an endoscope into a body cavity for examination and therapeutic purposes. The device is a tubular endless body that everts at its distal end and inverts at its proximal end when advanced through a duct such as the human colon. WO-A-99/01171 describes a similar device with the addition of corrugations designed to assist in the passage of the endless tube around the anatomy of the colon.

U.S. Pat. No. 5,941,815 describes a sigmoid splint device for use in endoscopy. The device is intended to be used to keep the sigmoid colon from looping while the operator is attempting to cross more difficult junctions.

In general, such known devices are either difficult to use, cause discomfort to the patient, can only be advanced incrementally, do not cater for complex nature and shape of tortuous body passageways.

There is a need for an improved medical device which will address at least some of these problems and which may be especially used as an introducer to navigate the lower gastrointestinal tract with minimum discomfort to the patient.

### STATEMENTS OF INVENTION

According to the invention there is provided a medical device for insertion in a body opening or an incision comprising:

a sleeve of pliable material having an outer sleeve section and an inner sleeve section;

a chamber for pressurised fluid defined between the inner and outer sleeve sections;

the inner sleeve section defining a lumen to receive an object,

the sleeve being evertable on engagement of an object in the lumen and axial movement of an object relative thereto so that the inner sleeve section is rolled over outwardly to become an outer sleeve section and the outer sleeve section is correspondingly rolled over inwardly to become an outer sleeve section;

at least portion of the sleeve having a non linear shape.

In a preferred embodiment of the invention the non linearity corresponds to a desired predetermined shape.

Preferably the non linearity is on at least two dimensions, usually in three dimensions.

The sleeve may be biassed into the non linear shape.

The sleeve may be sculpted or formed into the non linear shape.

In a particularly preferred embodiment of the invention the sleeve is turned axially back on itself to define an outer sleeve section and a twisted inner sleeve section.

Most preferably the sleeve sections define a continuous endless track which may be advanced by engaging an object in the lumen.

In one embodiment of the invention the device includes a guide collar for locating relative to a datum, a sleeve section being movable relative to the collar on engaging an object into the lumen and/or on passage of an object through the lumen. Typically, the fee ends of the sleeve ate joined to the collar.

In a preferred embodiment the device includes an inflation port for inflation of the enclosed chamber between the sleeve sections.

In one embodiment of the invention the devices includes guide means through which the sleeve and/or an object is advanced.

The guide means may include a ring means for placing in a body opening or incision through which the sleeve and/or an object is advanced.

In one embodiment of the invention the device includes a delivery means for delivery of the device to a remote location.

The delivery means may be a tube such as a catheter or a cannula.

In one aspect the device defines a transporter for delivery or retrieval of an object to or from a desired location.

In another aspect the device defines an introducer for introducing an object such as an instrument to a desired location.

In a further aspect the device is an expandable element such as a balloon for example for angioplasty.

### BRIEF DESCRIPTION OF DRAWINGS

The invention will be more clearly understood from the following description thereof given by way of example only, in which:

FIG. 1 is a perspective view from a proximal end of an introducer according to the invention.

FIG. 2 is a perspective view from a a distal end of the introducer of FIG. 1;

FIG. 3 is a cross sectional view of the introducer in one position of use;

FIG. 4 is a cross sectional view of the introducer in another position;

FIG. 5 is an end view of the introducer;

FIGS. 6 and 7 are diagrammatic view illustrating one method for manufacturing the introducer;

FIGS. 8 and 9 are side partially cross sectional view of other introducers;

FIGS. 10 to 14 are cross sectional views illustrating the operation of an expandable medical device according to the invention;

FIG. 15 is side, partially cross section view of the device of FIGS. 10 to 14, in use; and

FIG. 16 is an enlarged view of a detail of FIG. 15.

FIGS. 17 to 25 are diagrams illustrating the principles of operation of the invention.

FIG. 27 is a perspective view of an introducer device in an untwisted configuration;

FIG. 28 is a plan view of the device of FIG. 27;

FIG. 29 is a cross sectional view of the line A-A in FIG. 28;

FIG. 30 is a perspective view of the introducer device of FIG. 27 in a twisted configuration;

FIG. 31 is a plan view of the device of FIG. 30; and

FIG. 32 is a cross sectional view on the line B-B in FIG. 31.

### DETAILED DESCRIPTION

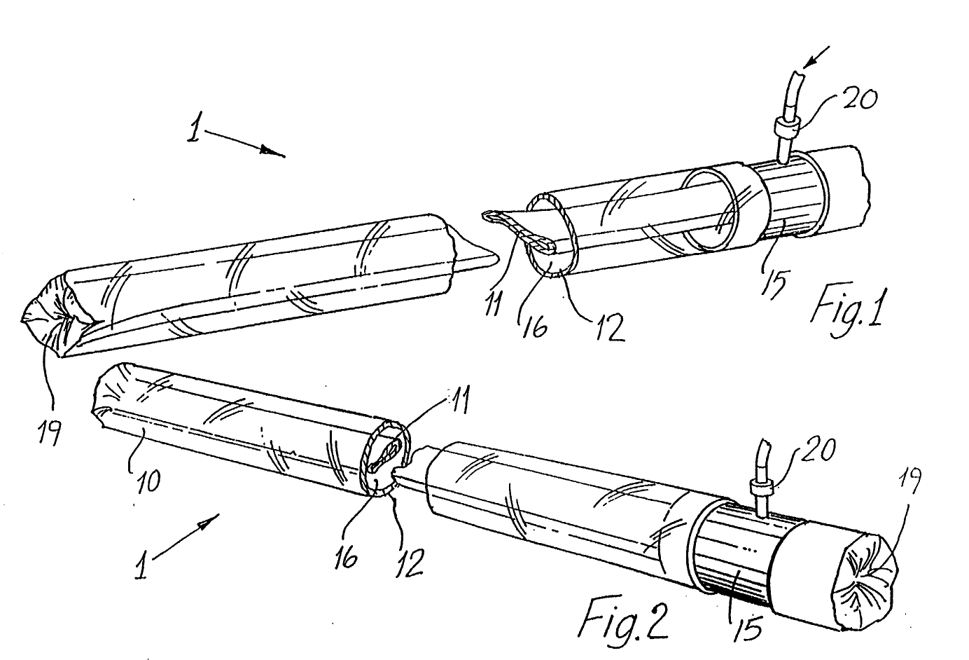

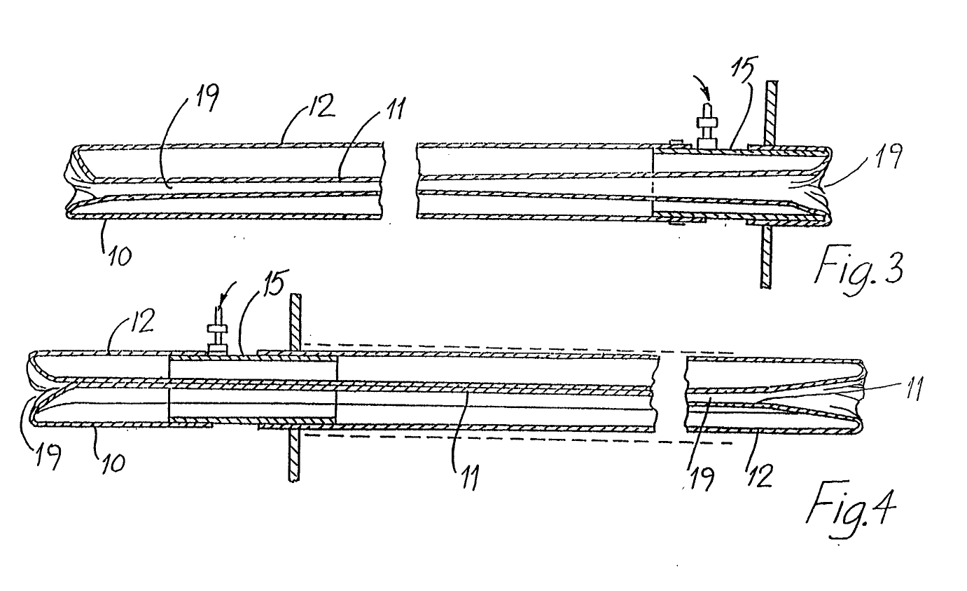

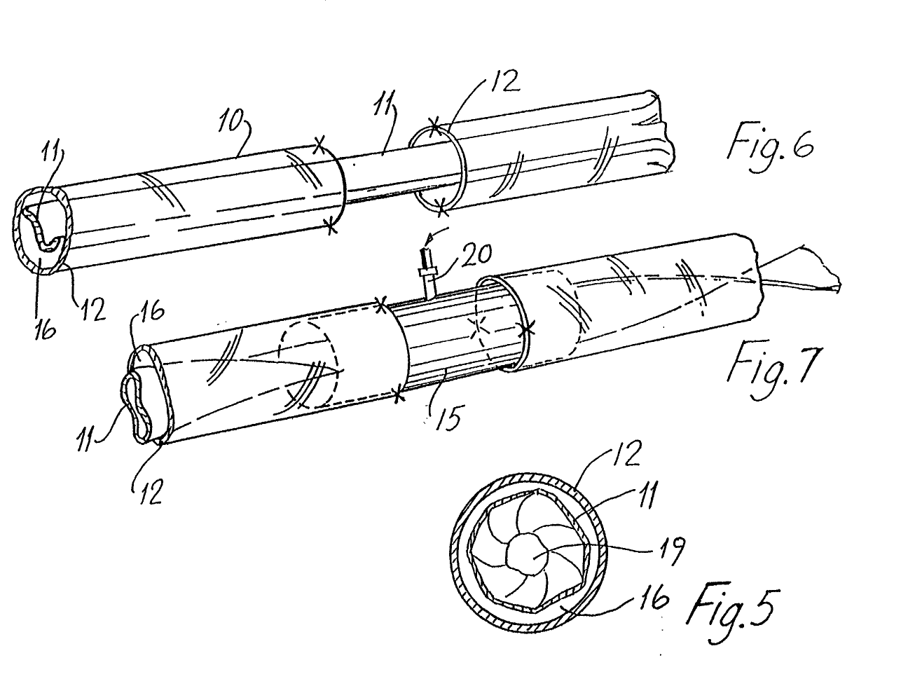

Referring initially to FIGS. 1 to 7 there is illustrated a medical device according to the invention which in this case is configured as an introducer 1 for introducing an object such as an instrument through a body opening such as the throat or rectum.

The device 1 comprises an elongate tubular sleeve 10 of pliable material, especially a suitable biocompatible gas impermeable plastics material which is turned axially back on itself to define an inner sleeve section 11 and an outer sleeve section 12. The sleeve sections 11, 12 are joined in this case via a collar 15, to define an enclosed inflatable chamber 16 therebetween. The inner sleeve section 11 defines an inner lumen 19 and the sleeve is twisted to centralise the lumen 19. An inflation port 20 is provided for inflating the chamber 16 between the inner and outer sleeve sections.

It will be particularly apparent that the device of the invention may be readily advanced through a complex passageway such as the bowel or the like. It may therefore be used for cannulating such a passageway.

FIGS. 6 and 7 illustrate the twisting of the sleeve 10. The free ends of the sleeve 10 are rotated relative to one another prior to or after final assembly to the collar 15. The twist will be apparent with reference to the points marked X. Such a twist may be provided when the device is in situ and is adjustable in situ.

The present invention provides a device that permits an endoscope or similar instrument to pass easily through the body's natural canals for purposes of performing an endoscopic examination of those canals. The introducer allows the user to easily pass beyond junctions such as the sigmoid colon, splenic and hepatic flexures and other convolutions of the body's inner canals in the upper or lower gastrointestinal tracts. In addition, the device allows pain free cannulation of the body's canals by substantially eliminating friction and rubbing contact between the endoscope and the walls of the canal under examination. Further the device is easy to manufacture and is convenient and simple to use.

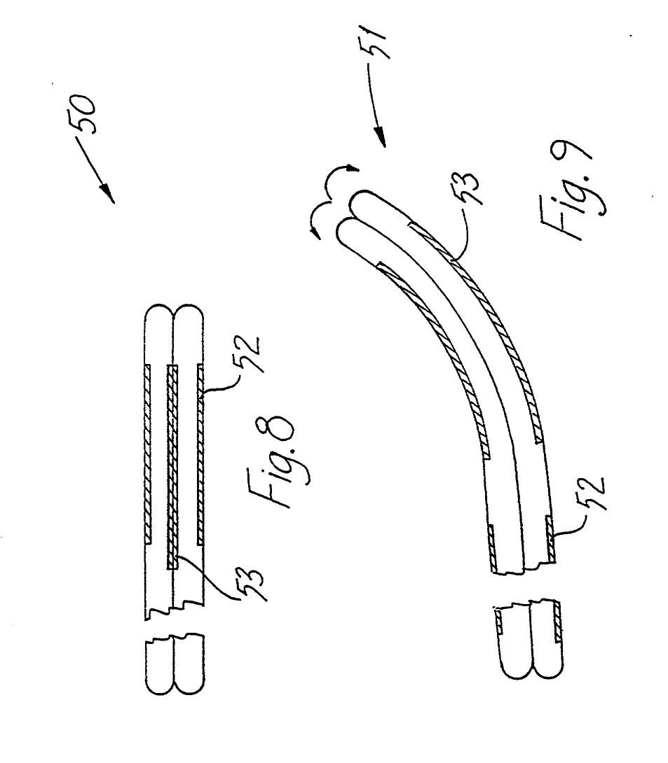

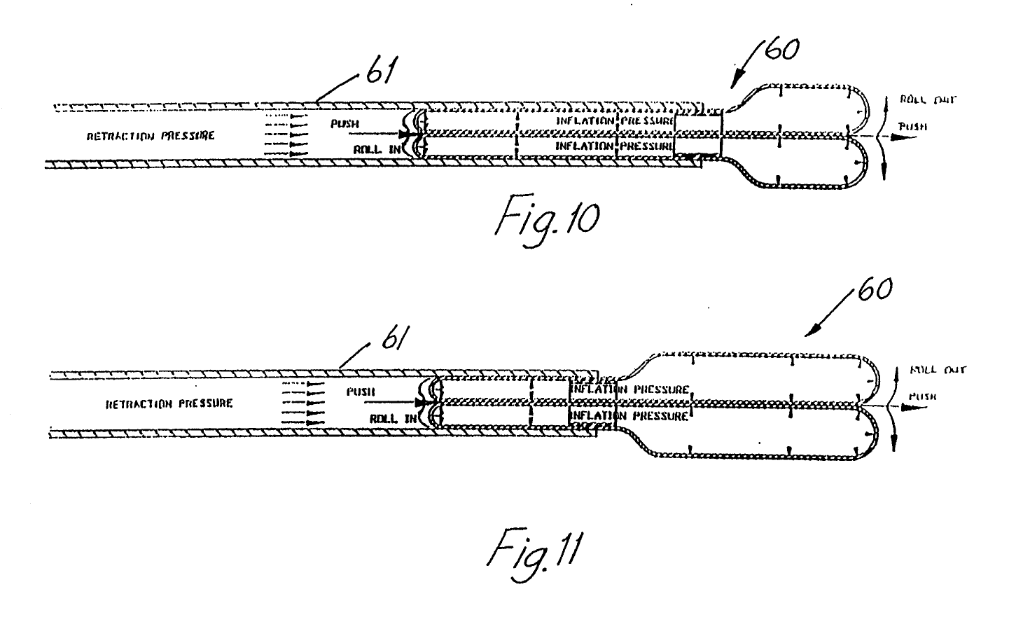

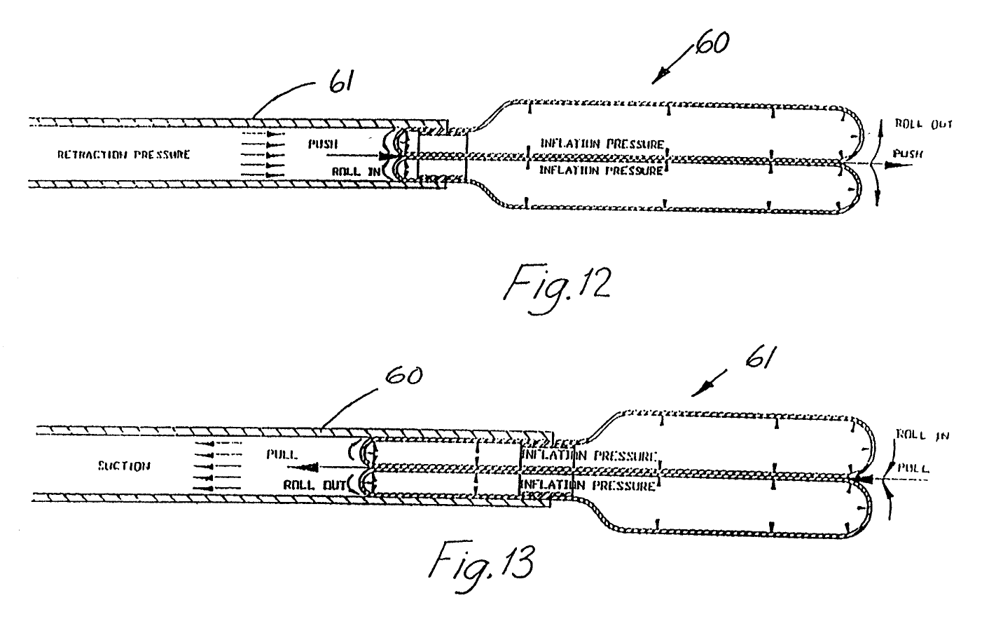

Referring to FIGS. 8 and 9 there are illustrated other devices 50, 51 according to the invention which may be twisted or not The devices are pre-shaped for a particular use. The devices may be biassed to form a desired non linear shape on eversion. In this case the device has sculpted sections 52, 53 to confirm, an eversion to a desired shape. It will be noted that the sculpted section 53 is initially part of the inner sleeve section. On eversion, it become unparalleled into a section of desired shape. The devices may be formed by molding and/or sculpting to define the desired shape dependent to the passageway to be navigated

The devices address two significant problems in lower GI endoscopy: the problem of friction between the endoscope and the colon and the problem caused difficulty in navigating bends and convolutions in the colon. Such a device would place itself between the colon and the endoscope such that the scope passed through the lumen of the colon without touching the side walls of the colon. Some conventional introducers can address this problem, especially in a straight section of colon, but will have difficulties when the colon is in any way convoluted or has a tight junction or bend. In reality all colons have such a twisted layout. In the case of conventional devices the device may fail to tun at the bend and will simply push up against the side wall of the colon causing pain and possibly damage to the mesentry. In serious cases the wall of the colon may be perforated.

The devices of the invention are predisposed to bend at certain points or gradually slope in a certain direction. In this way the device, for example, can be inserted into the rectum and will be disposed towards following the bend of the sigmoid colon or turn at the splenic flexure as it moves through the colon. The device may be predisposed to bend at more than one point. For example, it could be constructed in such a manner that it would gradually curve through the sigmoid colon and then straighten out to traverse the descending colon. A further turn could be constructed into the device so that it would turn at the splenic flexure. By preshaping the everting tube in this manner it would be possible to plan all the bends and convolutions in a passageway to be navigated.

The device may be used as a transporter for delivery or retrieval of an object. It has the effect of providing a substantially frictionless tunneling action. The device may be used endoluminally. The device may be used for example to provide a soft tissue dissector or an envaginator and may be delivered through a delivery device such as a tubular sleeve, catheter or the like. The device itself may be used in medical procedures such as in the form of a balloon which may be linear or non linear.

Referring to FIGS. 10 to 16 there is illustrated a device 60 similar to that of FIGS. 1 to 7 which is deliverable through a tube 6 such as a cannula. A pressure is applied to push the device 61 out through an end opening of the tube 61 as illustrated in FIGS. 10 to 12. To retract the device a suction force is applied to draw the device 60 back into the tube 61 as illustrated in FIGS. 13 and 14.

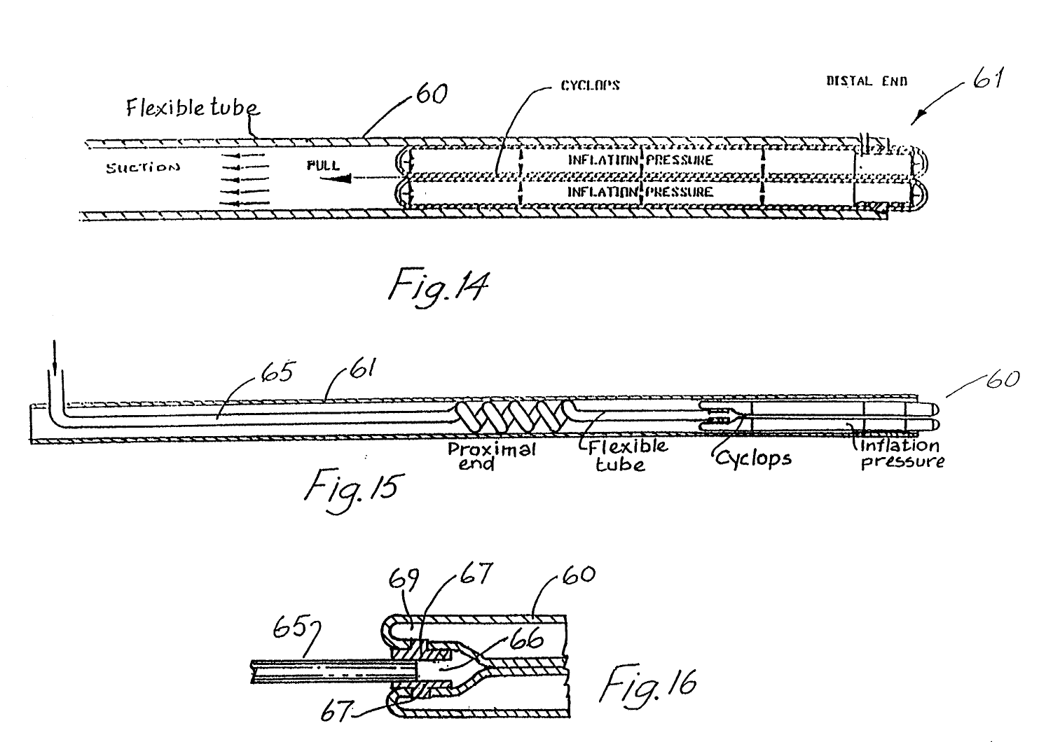

Referring in particular to FIGS. 15 and 16 there is illustrated one mechanism which may be used to deliver the device 60 through the tube 61 and to remotely control the operation of the device 60. Air is delivered through an air delivery tube 65 extending through the outer tube 61. The air delivery tube 65 has a central outlet 66 for driving the device 60 and one or more entry ports 67 for delivery of inflation air into the air chamber 69 defined by the device 60. In this way the inflation of the device 60 can be readily remotely controlled. The device 60 may be used, for example for tissue dissection or for delivery/deployment of a balloon angioplasty, stent or the like.

### The Twisted Tube

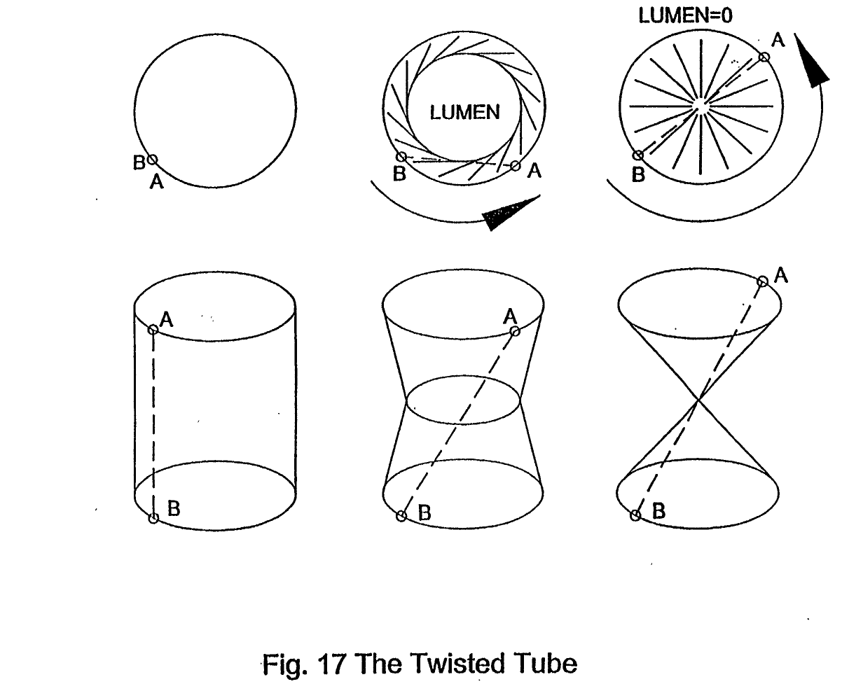

Consider the hollow cylindrical tube shown in FIG. 17. The wall of the cylinder defines a lumen through its center. Consider a linear element A-B, if the upper edge of the tube is rotated through some angle point A will move to the position shown in the middle sketch. The element A-B will still define a straight line. The tube will distort into a nominally hour glass shape with a reduced lumen at mid height. The diameter of the lumen at the neck of the tube is dependant on the angle of twist. When the upper edge is rotated through 180° the lumen will close down to zero diameter. At any horizontal plane through a twisted tube the material must be wrinkled and hence under compressive hoop stress. If the height of the tube remains unaltered then the element A-B in a twisted tube, being longer than in a plain tube, must be under tensile axial stress. If the tube is free of axial constraint the overall length of the tube will reduce,

### Lumen Diameter vs. Angle of Twist

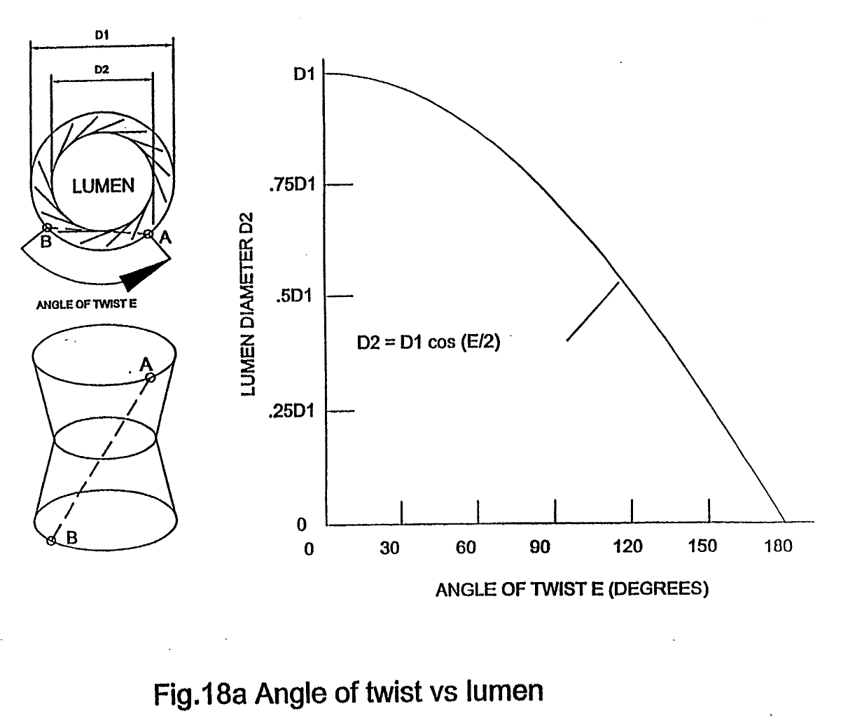

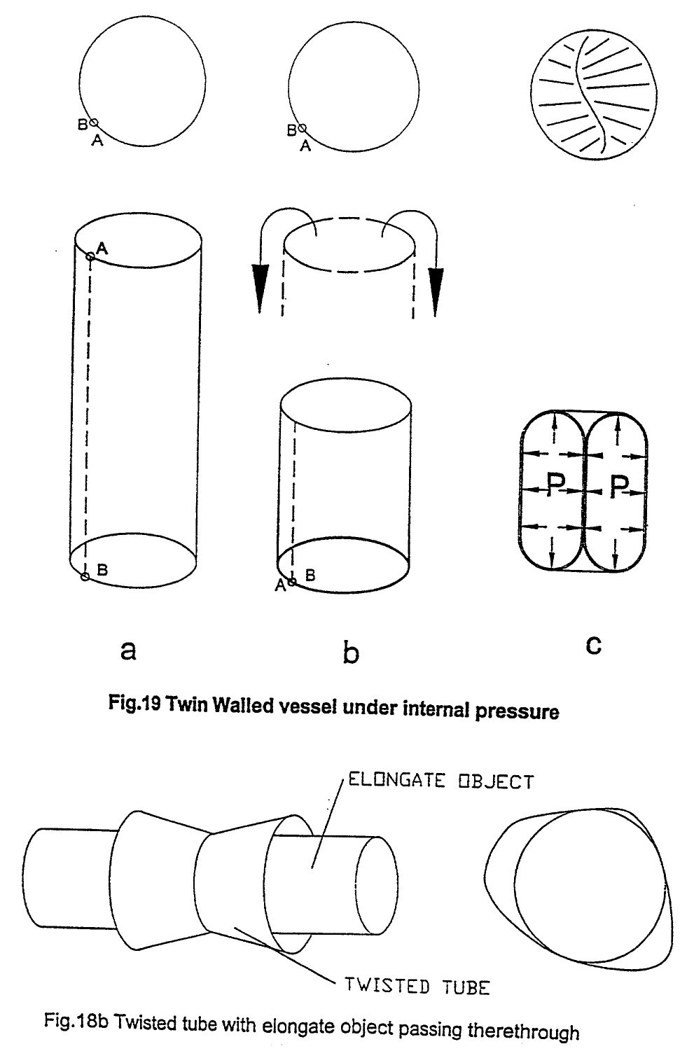

FIG. 18 a shows the lumen diameter (D2) as a proportion of the tube diameter (D1)for angels of twist (E) from 0 to 180 degrees. The lumen diameter is calculated as:

D2=D1 cos(E/2).

As can be seen the lumen diameter is independent of the tube length

### Elongate Object Passed Through Twisted Tube

As can be clearly seen from FIGS. 18a and 18b the angle of twist necessary to collapse the lumen of a tube to the diameter of an elongate object passed therethrough is dependent on the ratio of the tube diameter and the diameter of the elongate object. The angle of twist can be calculated from:

Cos.⁻¹(E/2)=D2/D1

Where E=angle of twist

D1 =tube diameter

D2=diameter of elongate object

Although depicted as of circular profile, a tube of sufficiently compliant material will conform to any non recursive profile. For such a profile D2 is taken as the smallest diameter which can be inscribed within the profile.

### Twin Walled Pressure Vessels Under Internal Pressure

Consider a thin walled tube as shown in FIG. 19a. One end of the tube is folded back on itself as shown in FIG. 19b and the fee ends conjoined. What is defined is essentially a twin walled tube (or two coaxial tubes conjoined at their ends) with an enclosed volume between the two walls. The introduction of a pressurised fluid into die enclosed volume will cause the outer tube to behave like a pressurised aircraft fuselage, that is it will be subject to tensile axial and hoop stresses. The inner tube will be subject to tensile axial stress and compressive hoop stress. As a result the lumen will collapse in to a nominally duck bill configuration but constrained by the outer tube.

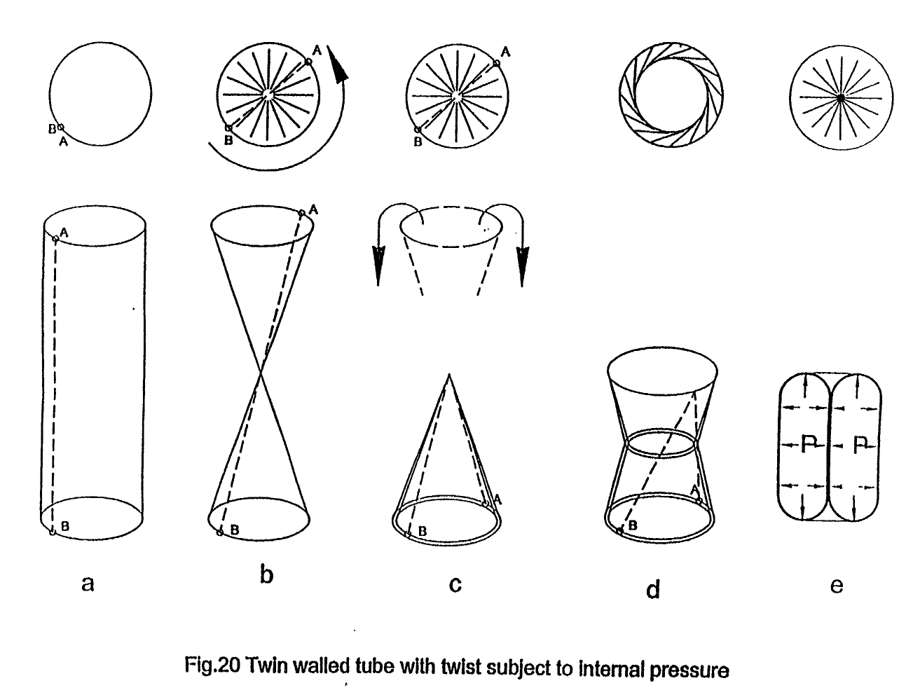

Greater control of the lumen can be obtained by the introduction of a twist into the tube. The tube shown in FIG. 20a is twisted as shown in FIG. 20b. One end of the tube is folded back on itself, as shown in FIG. 20c, and the free ends conjoined. As in the previous example this configuration defines two coaxial conical vessels conjoined at their bases and at the apex. However the common apex is not constrained to remain in this configuration. In reality in the inner and outer tubes are free to behave as individual tubes each with half of the original twist and as such the composite tube can better be defined as two coaxial hour glass tubes as shown in FIG. 20d, each containing half the original total twist. As both the inner and outer tubes are necked they each are subject to compressive hoop stresses. The introduction of a pressurised fluid into the enclosed volume produces what is believed to be a novel response.

Firstly, consider the outer tube. It is a necked hour glass tube with compressive hoop stresses. The introduction of the pressurised fluid induces tensile hoop stresses, negating the compressive hoop stresses induced by the twist. Since, to remain in its twisted configuration, the tube must have compressive hoop stresses and since the pressurised fluid overcomes these compressive stresses the tube must untwist and take on a nominally cylindrical configuration, see FIG. 20e. Since the inner and outer tubes are conjoined, as the outer tube untwists the inner tube twists more in response. Since the outer tube now has no twist the inner tube must have all the twist. If the original total twist were 180° then the lumen would close totally. Additionally, the material defining the inner tube will be central within the diameter of the outer tube. This configuration will for brevity be called a Cyclops.

### Translation of an Elongate Object Through a Cyclops

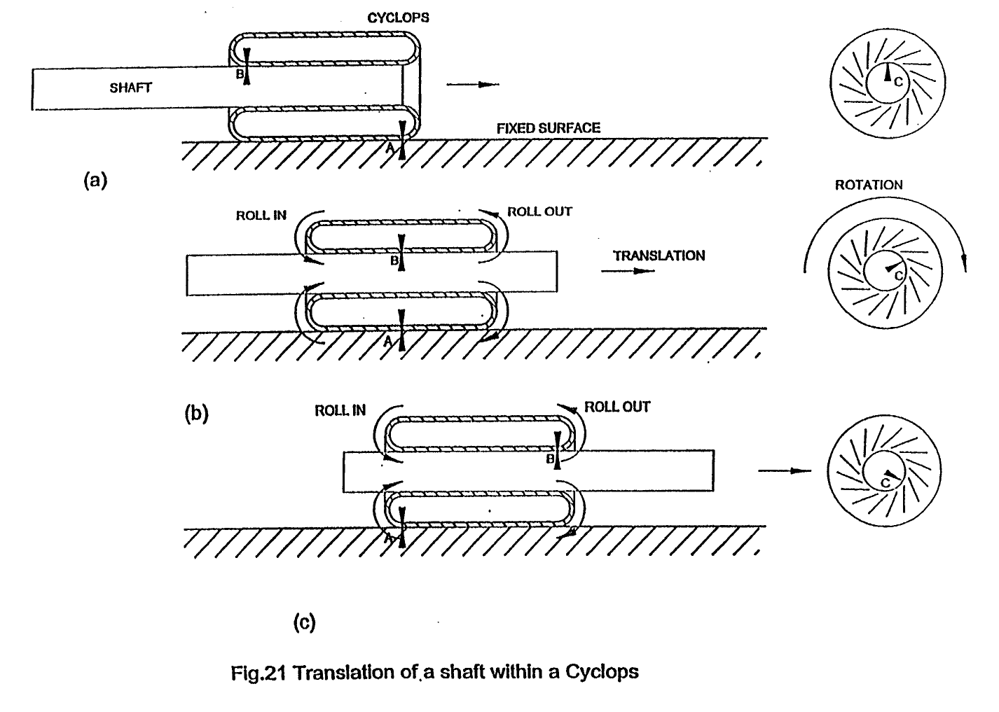

Consider the arrangement depicted in FIG. 21a. A shaft is passed through a Cyclops with the lumen in mutual contact with the shaft. The outside diameter of the Cyclops is resting in mutual contact with a fixed surface. Consider points of contact A, between the Cyclops and the fixed surface, and B, between the shaft and the lumen of the Cyclops. As the shaft is translated, as shown in FIG. 21b, Point A remains fixed whilst the leading end of the lumen rolls out. Since the Cyclops does not change in overall length the trailing end of outside diameter rolls in as depicted. It will be apparent that the shaft translates to the right twice as far as the Cyclops. This, of course, is exactly the motion of a caterpillar track. From this point of view a Cyclops could be considered as a three dimensional caterpillar track. Since points A and B on the Cyclops do not move relative to their corresponding positions on the shaft and the fixed surface there is no frictional resistance to the translation of the shaft. In FIG. 21c the Cyclops has translated to the right by approximately its own length. The material which had originally formed the inner tube has rolled out to become the outer tube and vice versa. In other words the Cyclops has turned inside out. Since the inner tube of the Cyclops is in a twisted configuration and since the point B remains in contact with the same point on the shaft the shaft rotates about its axis as depicted by arrow C (in this instance approx. 120°). In order to obtain this translation the resistance required to be overcome is that generated as the leading and trailing ends of the Cyclops deform as they roll out and roll in respectively.

### Effects of an Introduced Member

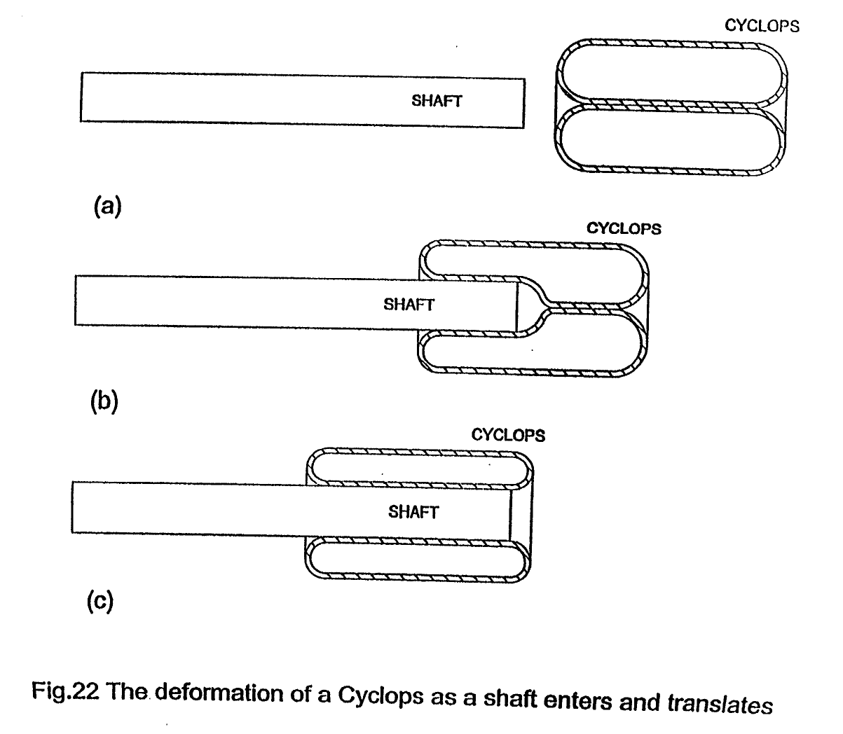

Assume that the Cyclops in FIG. 22 has a 180° twist and is filled with a fluid under pressure. The lumen is closed by the action of the twist. As the shaft enters the Cyclops the pliable nature of the material of the Cyclops allows it to envelop the leading edge of the shaft as shown in FIG. 22b. As can be seen the effective volume of the Cyclops decreases. There will be a resulting increase of the fluid pressure causing the lumen to exert a greater pressure on the shaft. As the shaft proceeds through the Cyclops, see FIG. 22c, the pressure increases to it's maximum. (P1V1=P2V2). If it is not desirable that there be such a pressure increase then the supply of fluid could be controlled by a pressure regulator.

### Forces Acting on the Lumen due to Axial Component of Pressure

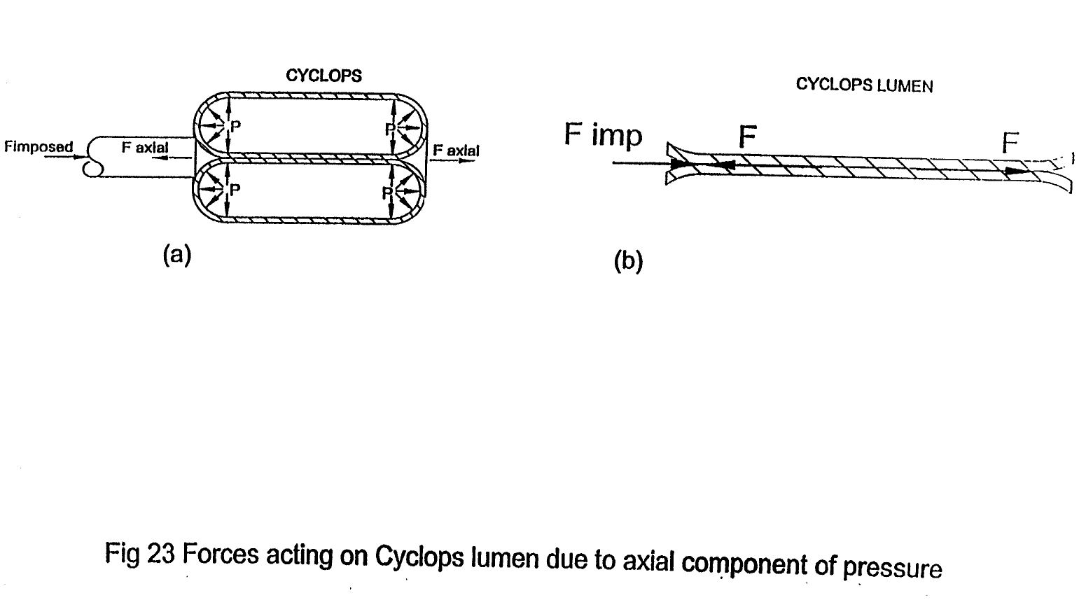

FIG. 23a depicts a Cyclops subject to internal pressure P. A force (Faxial) is induced in the Cyclops. Since the cross sectional area of the Cyclops is uniform the system is in force balance. A proportion of this force is taken by the outside cylinder and the remainder is taken by the material which constitutes the lumen. If a shaft, or similar, is pressed against one end of the Cyclops an imbalance is introduced. Consider the material of the inner tube in isolation. FIG. 23b, F represents the proportion of the axial force taken by the inner tube. As with the Cyclops as a whole this is in force balance. When an imposed force (Fimp) is applied to one end of the inner tube the net force acting at the left end of the tube is now less than that acting on the right hand end. The system is in an unbalanced situation. The inner tube must therefore translate to the right hand side.

### Cyclops as an Axial Force Limiter

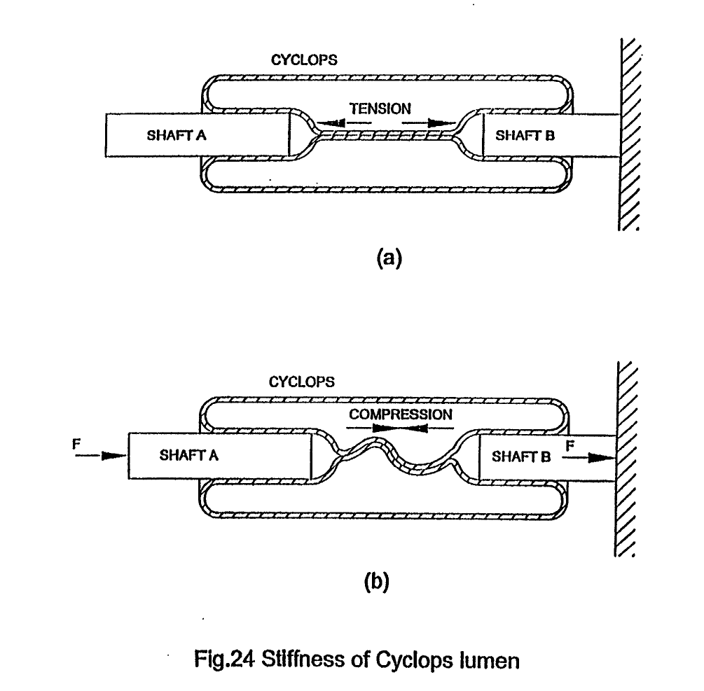

FIG. 24a depicts a Cyclops with two independent shafts inserted, one in each end. Shaft B is in contact with a fixed surface. The fluid pressure within the Cyclops causes the lumen material to be maintained in tension. A force F is applied to shaft A, FIG. 24b. This force is transferred to shaft B via the lumen material and is reacted by the fixed surface. Force F exerts a compressive axial force on the lumen material. As force F increases the applied compressive force begins to negate the axial tensile force induced by the pressurised fluid. When force F becomes greater than the initial tensile force the lumen material goes into axial compression. This will cause the lumen material to buckle. The maximum axial force that the inner tube of a Cyclops can transmit is equal to the tensile force induced into the inner sleeve by the pressurised fluid.

### Effects of the Tube Preform Shape

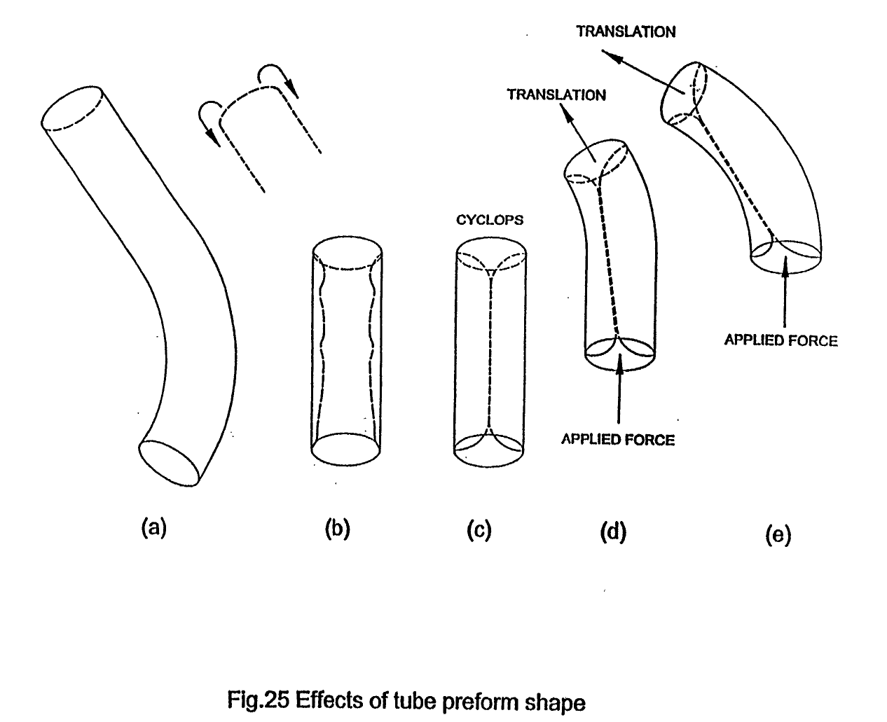

Consider the tube preform shown in FIG. 25a. The lower portion defines a circular elbow with an upper section configured as a plain cylinder. If the cylindrical section is inverted as indicated, with or without a twist, and the free ends of the preform conjoined a basic Cyclops is formed. The elbow section must wrinkle up in order to lie within the plain cylinder as shown in FIG. 25b. As previously, the introduction of a pressurised fluid into the enclosed cavity formed will cause the elbow section to collapse forming a closed lumen and the cylindrical section to inflate, see FIG. 25c. The plain cylinder, being on the outside of the structure, will determine the shape of the inflated Cyclops. The Cyclops will be in force balance. If a force is applied to the lower end of the lumen the net force on the lumen will cause the Cyclops to translate. The upper portion of the lumen will roll out and the lower end of the cylindrical wall will roll in, FIG. 25d. Since the outside wall of the Cyclops is now made up of part of the original plain cylinder and part of the elbow section the inflation pressure will cause the Cyclops to take on the form of this composite profile, the Cyclops will appear to bend as it translates. FIGS. 25d shows the Cyclops completely inverted. All of the elbow section forms the outer wall and all of the plain cylindrical section forms the inner tube. As such the Cyclops has take on the form of the elbow section. It will be noted that lumen material follows the shortest line between the ends of the Cyclops. For simplicity of illustration the Cyclops has been represented as translating in two space. It will be apparent that if the preform were “sculpted” or molded such that its axis were three dimensional then as the Cyclops translated the path of translation would follow a three dimensional path. It will also be appreciated that the preform need not be of a regular cross section. Variations in tube diameter along it's length is possible.

Referring to FIGS. 26(a) to 26(d) there is illustrated the roll-out or eversion of a pre-shaped device, in this case an introducer device 100. The device 100 is in this case pre-shaped or sculpted to roll-out in a non-linear shape corresponding to a desired predetermined shape. The non linearity may be in two, and in this case three dimensions. The introducer 100 is initially in the shape of a tubular sleeve. On eversion the introducer first turns in one direction (FIG. 26(b)), then in another direction (FIG. 26(c)) and, finally in a still further direction illustrated in FIG. 26(d). Such an arrangement greatly facilitates the navigation of the device along tortuous routes.

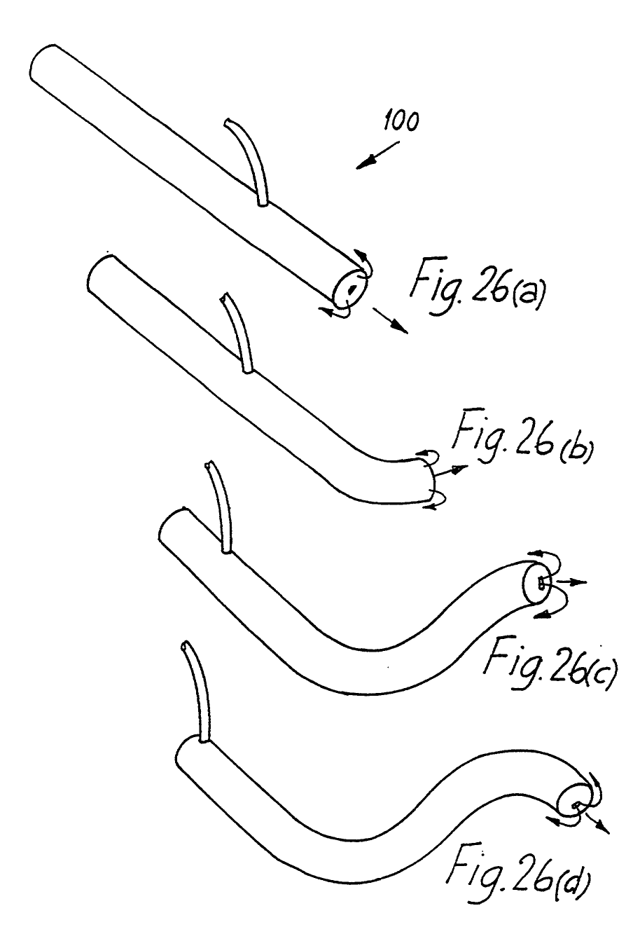

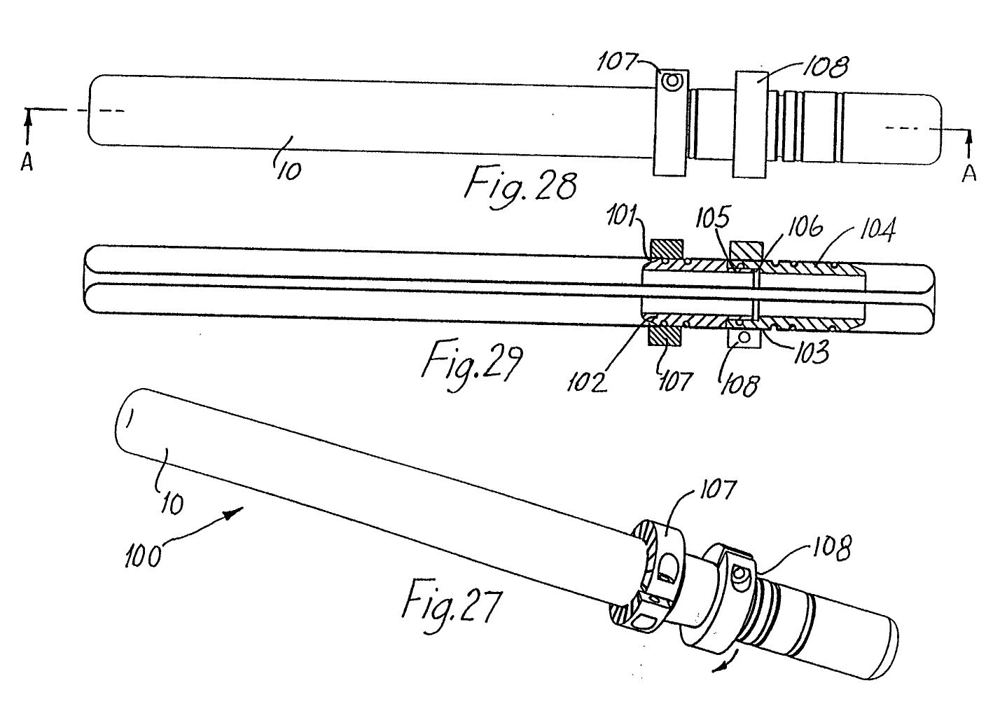

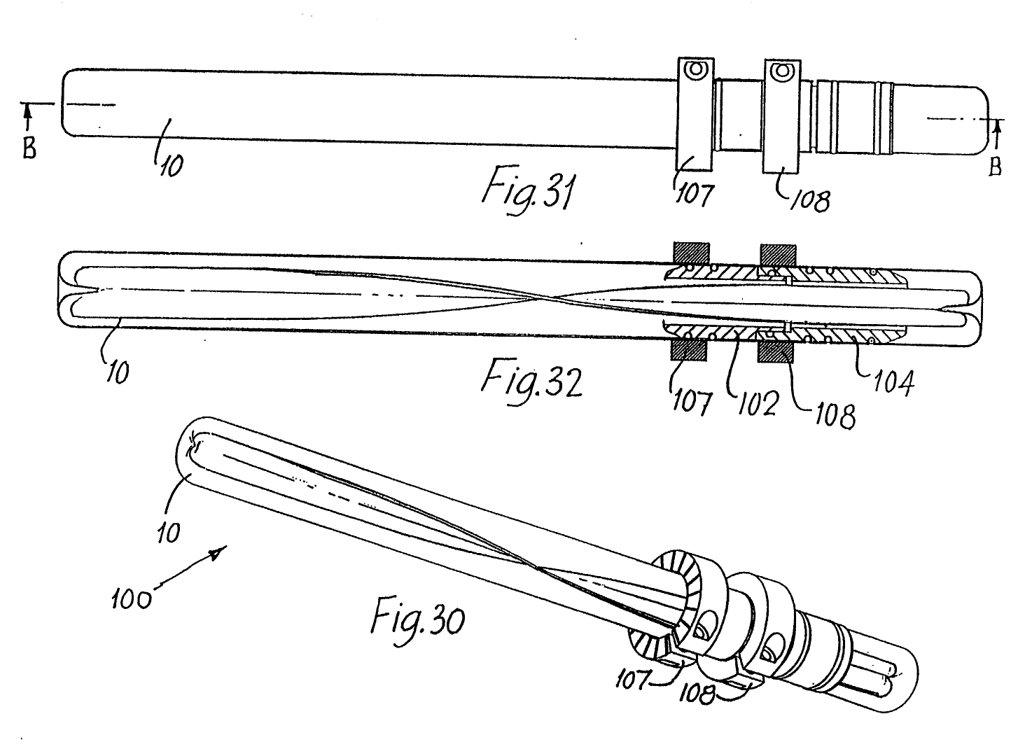

Referring to FIGS. 27 to 32 there is illustrated another introducer device 100 according to the invention which is similar to the devices described above and like parts are identified by the same reference numerals. In particular, the device 100 is similar to and operates in a similar manner to the introducer described above with reference to FIGS. 1 to 7. In this case the introducer is adjustable to vary the degree of twist in the elongate tubular sleeve 10 from an untwisted configuration illustrated in FIGS. 27 to 29 to a twisted configuration illustrated in FIGS. 30 to 32.

The sleeve 10 is attached at one end 101 to a first shaft section 102 and is attached on the other end 103 to a second shaft section 104. The shaft sections 102, 104 have a respective male projection 105 and a complementary female recess 106 which interengage to facilitate relative rotation therebetween. The shaft sections 102, 104 have respective handles formed by knobs 107, 108 to facilitate manipulation to vary the degree of twist. In use, the shaft section 104 may be turned by the knob 108 to any desired extent, for example from the untwisted configuration of FIG. 27 to the twisted configuration of FIG. 30. The variation in the twist may be effected prior to insertion of the device and/or when the device is in situ.

Reference is also made to appropriate alternatives and modifications which are outlined in our parallel applications referenced ATRO1/C, ATRO12/C, ATRO14/C/, ATRO15/C, ATRO17/C, the entire contents of which are incorporated herein by reference.

The invention is not limited to the embodiments hereinbefore described which may be varied in detail.

1. A medical device for insertion in a body opening or an incision comprising:
a sleeve of pliable material having an outer sleeve section and an inner sleeve section;
a chamber for pressurised fluid defined between the inner and outer sleeve sections;
the inner sleeve section defining a lumen to receive an object;
the sleeve being evertable on engagement of an object in the lumen and axial movement of an object relative thereto so that the inner sleeve section is rolled over outwardly to become an outer sleeve section and the outer sleeve section is correspondingly rolled over inwardly to become an outer sleeve section;
at least portion of the sleeve having a non linear shape.

2. A device as claimed in claim 1 wherein the non linearity corresponds to a desired predetermined shape.

3. A device as claimed in claim 1 or 2 wherein the non linearity is on at least two dimensions.

4. A device as claimed in any of claims 1 to 3 wherein the non linearity is in three dimensions.

5. A device as claimed in any of claims 1 to 4 wherein the sleeve is biassed into the non linear shape.

6. A device as claimed in any of claims 1 to 5 wherein the sleeve is sculpted or formed into the non linear shape.

7. A device as claimed in any preceding claim wherein the sleeve is turned axially back on itself to define an outer sleeve section and a twisted inner sleeve section.

8. A device as claimed in any preceding claim wherein the sleeve sections define a continuous endless track which may be advanced by engaging an object in the lumen.

9. A device as claimed in any preceding claim including a guide collar for locating relative to a datum, a sleeve section being movable relative to the collar on engaging an object into the lumen and/or on passage of an object through the lumen.

10. A device as claimed in claim 9 wherein the free ends of the sleeve are joined to the collar.

11. A device as claimed in any preceding claim including an inflation port for inflation of the enclosed chamber between the sleeve sections.

12. A device as claimed in any preceding claim including guide means through which the sleeve and/or an object is advanced.

13. A device as claimed in claim 12 wherein the guide means includes a ring means for placing in a body opening or incision through which the sleeve and/or an object is advanced.

14. A device as claimed in any preceding claim including a delivery means for delivery of the device to a remote location.

15. A device as claimed in claim 14 wherein the delivery means is a tube.

16. A device as claimed in claim 14 or 15 wherein the delivery means is a catheter.

17. A device as claimed in any preceding claim wherein the device defines a transporter for delivery or retrieval of an object to or from a desired location.

18. A device as claimed in any preceding claim wherein the device defines an introducer for introducing an object such as an instrument to a desired location.

19. A device as claimed in any of claims 1 to 17 wherein the device is an expandable element.

20. A device as claimed in claim 19 wherein the expandable element is a balloon for angioplasty.

21. A medical device substantially as hereinbefore described with reference to the accompanying drawings.
